(19) United States
(12) Patent Application Publication
(10) Pub. No.: US 2011/0064717 A1
(43) Pub. Date: Mar. 17, 2011

(54) Homing endonuclease genes and their targets

(75) Inventors: Adi Barzel, Moshav Nir-Tzvi (IL); Eyal Privman, Yavne (IL); Michael Pe'eri, Tel Aviv (IL); Martin Kupiec, Tel Aviv (IL); Tal Pupko, Herzelia (IL)

(73) Assignee: Ramot at Tel-Aviv University Ltd., Tel Aviv (IL)

(21) Appl. No.: 12/806,806

(22) Filed: Aug. 20, 2010

Related U.S. Application Data
(60) Provisional application No. 61/274,789, filed on Aug. 20, 2009.

(51) Int. Cl. A61K 38/46; A61K 31/7088; A61P 19/10; A61P 35/00; A61P 27/02; C12N 15/74; C12P 19/34; C12N 1/20

(52) U.S. Cl. 424/94.6; 514/44 R; 435/471; 435/91.1; 435/252.1

(57) ABSTRACT

The invention provides pharmaceutical composition having as an active ingredient either a homing endonuclease (HE) capable of cleaving a non-native target nucleotide sequence in a genome or a nucleotide sequence encoding for a HE capable of cleaving a target site in a non-native genome. The invention also provides uses for such HEs, and methods of treatment utilizing such HEs. The HE may be, for example, any one of the HEs PI-SceI, POLB HE, PRP8 HE, or Nostoc species PCC7120 HE.

### CROSS-REFERENCE TO RELATED APPLICATIONS

This application claims the benefit of the filing date of U.S. Provisional Patent Application No. 61/274,789, filed Aug. 20, 2009, the disclosure of which is hereby incorporated herein by reference.

### FIELD OF THE INVENTION

This invention relates to DNA endonucleases.

### BACKGROUND OF THE INVENTION

Gene therapy aims to cure diseases by treating their genetic basis rather than their manifestations. It entails the delivery of corrective genes into affected cells in order to replace, inhibit, correct or compensate for the expression of a disease causing allele. The great promise of gene therapy is to provide a remedy for illnesses that are otherwise difficult to address, such as congenital genetic disorders, neurodegenerative diseases, viral infections and cancer. However, after years of research, two main challenges still stand in the way of wide and successful gene therapy applications. First, the vector carrying the corrective gene must be delivered to the appropriate tissues or cell types and only to them, in order to avoid toxic side effects. Second, when the corrective gene has entered the cell, it must be expressed in a controlled manner, and without disturbing the due expression of other important genes. Controlled expression can best be achieved either by correcting the mutated gene at its native location, or by inserting the transgene at a safe genomic harbor, an intergenic region far away from any gene in general and any possible oncogene in particular. This form of precise correction or safe complementation is called gene-targeting. In addition to the above medical utilities, gene targeting can also be used for biotechnological enterprises such as crop improvement and for research undertakings such as the engineering of knockout mice strains that allow scientists to model human diseases and test potential remedies.

Transfection of human cells by vectors carrying a corrective gene very rarely results in gene targeting. These rare events are attributed to spontaneous homologous recombination (HR) between the vector-borne gene and the endogenous allele. There are several ways to increase the rate of HR; by far the most effective of which is the induction of a site specific double strand break (DSB). Such DSBs have been shown to raise the frequency of gene targeting by as much as five orders of magnitude. However, induction of a unique DSB is challenging due to the sheer size of the human genome (about 3*10⁹ base pairs (bp)). For example, a restriction enzyme with an 8 bp long target sequence will cleave the human genome approximately 3*10⁹/4⁸≈45,776 times. Such excessive or non-specific cleavage may result in cell death or worse, in genomic instability leading to malignant transformation. There are two major approaches to the challenge of introducing unique DSBs into the human genome. The first approach entails the design of chimeric proteins consisting of a non-specific endonuclease domain linked to a combination of DNA binding domains; the latter typically being zinc finger domains and the chimeras being zinc finger nucleases or ZFNs. ZFNs have been shown capable of inducing gene targeting in human cells. However, much concern has been raised regarding their possible toxicity.

The alternative approach advocates the use and manipulation of naturally occurring site-specific DNAases having long target sequences, namely homing endonucleases or HEs. HEs are a large and diverse class of site-specific DNAases found in Archaea, Eubacteria and lower eukaryotes, and in their respective viruses. The lengths of HE target sequences range between 14-40 bp. Furthermore, these targets are not stringently defined. Cleavage is tolerant to some base-pair substitutions along the target sequence. This has raised hopes that at least some HEs can introduce unique DSBs in desired loci of the human genome. However, only a few hundred HE genes (HEGs) have been annotated to date, and only a few dozen of which have been experimentally characterized. The chances are therefore slim for finding within this limited collection a HE suitable for gene targeting of a desired gene. One possible way to circumvent this limitation is by attempting to shift the target specificity of a given HE to make it capable of cleaving a desired sequence (e.g. one that is found within a disease related gene). This has been done with considerable success using a combination of directed enzyme evolution and rational design. Engineered HEs have been manufactured capable of cleaving XPC (deficient in Xeroderma Pigmentosum), IL2RG (deficient in X-linked SCID-severe combined immunodeficiency), Rag1 (deficient in autosomal recessive SCID) and the tumor suppressor gene p53. Despite its achievements, HE-engineering is an inherently limited approach; using directed evolution and rational design one can only alter target specificity up to a certain extent. Therefore, for HE mediated gene targeting to become a common medical practice, the arsenal of target sites must be dramatically extended by the discovery of many more naturally occurring HEs.

HEs have been utilized in gene targeting procedures where the introduction of site-specific double-strand-breaks facilitates gene correction, disruption or insertion at a locus of choice³. U.S. Pat. Nos. 6,528,313 and 6,528,314, European patent EP 419 621 and Japanese patents JP 3059481, JP 3298842 and JP 3298864 disclose use of homing endonucleases in gene targeting. WO2009/101625 discloses methods for searching for endonucleases in a database of sequences.

### DESCRIPTION OF THE INVENTION

The present invention is based on the novel and unexpected finding of homing endonucleases (HEs) capable of cleaving a target nucleotide sequence in the human genome as well in the genome of various animals. Thus, in its first aspect, the present invention provides pharmaceutical compositions comprising a HE or a nucleotide sequence encoding an HE capable of cleaving a non-native target sequence together with a pharmaceutically acceptable carrier. The nucleotide sequence may be, for example, a DNA sequence or an RNA sequence.

In one embodiment, the pharmaceutical composition comprises the HE PI-SceI HE from the yeast S. cerevisiae, which has the amino acid sequence SEQ ID No. 2 and is encoded for by a S. cerevisiae gene having the nucleotide sequence SEQ ID No. 1. The inventors have found that PI-SceI HE is capable of cleaving a target site located in the human ATP6V1A1 gene which encodes for a subunit of a lysosomal H⁺-ATPase, as well as homologous target sequences in several animal genomes. Inhibitors of lysosomal H⁺-ATPases have been used in the treatment of osteoporosis. Thus, a pharmaceutical composition of the invention comprising PI-SceI HE can be used in the treatment of osteoporosis.

In another embodiment of the pharmaceutical composition of the invention, the pharmaceutical composition of the invention comprises a HE from H. volcanii referred to herein as “POLB HE”. POLB HE is a HE encoded within an intein of the gene encoding for the DNA polymerase β of H. volcanii. POLB HE has the amino acid sequence SEQ ID 12 and is encoded by the DNA sequence SEQ ID 11. The inventors have found that POLB HE is capable of cleaving a target nucleotide sequence in the human POLD1 gene. Mutations in the human POLD1 gene have been associated with colon cancer and colorectal cancer. Thus, a pharmaceutical composition of the invention comprising POLB HE can be used in the prevention and treatment of cancer, and in particular colon cancer and colorectal cancer.

In a third embodiment of the pharmaceutical composition of the invention, the pharmaceutical composition of the invention comprises the HE from B. cinerea “PRP8 HE” which has the amino acid sequence SEQ ID No. 16 and is encoded by the DNA sequence SEQ ID No. 15. The inventors have found that PRP8 HE is capable of cleaving a target nucleotide sequence in the human PRPF8 gene. Several different mutations are known in the human PRPF8 gene that have been associated with the progressive blinding disease retinitis pigmentosa. Thus, a pharmaceutical composition of the invention comprising PRP8 HE can be used in the prevention and treatment of retinitis pigmentosa.

In another of its aspects, the invention provides use of the pharmaceutical composition of the invention for the treatment of a disease. The disease may be, for example, osteoporosis, cancer, in particular colon cancer and colorectal cancer, and retinistis pigmentosa.

In still another of its aspect, the invention provides a use of a HE to cleave a DNA sequence. In accordance with the aspect of the invention, an HE capable of cleaving a non-native nucleotide sequence in a genome is used to manipulate a DNA sequence whose amino acid translation has at least 80% homology with the amino acid translation of the native target of the HE with the proviso that the DNA sequence is not the native nucleotide sequence.

The HE may be, for example, PI-SceI HE, POLB HE, PRP8 HE, or Nostoc RNR, which has the amino acid sequence SEQ ID No. 21 and is encoded by the DNA sequence of SEQ ID No. 20.

Thus, in one embodiment, the invention provides a pharmaceutical composition comprising as an active ingredient either a homing endonuclease (HE) capable of cleaving a non-native target nucleotide sequence in a genome or a nucleotide sequence encoding for a HE capable of cleaving a target site in a non-native genome, together with a physiologically acceptable carrier.

The active ingredient of the pharmaceutical composition of the invention may be, for example, PI-SceI having the amino acid sequence SEQ ID No 2, POLB HE having the amino acid sequence SEQ ID NO. 12, or PRP8 HE having the amino acid sequence SEQ ID No. 16 amino acid. The active ingredient may be a DNA sequence or an RNA sequence.

The active ingredient of the pharmaceutical composition of the invention may be a DNA sequence, for example, any one of the DNA sequences SEQ ID No. 1, SEQ ID No. 11, or SEQ ID No. 15. The active ingredient of the pharmaceutical composition of the invention may be an RNA sequence, for example, any one of the RNA sequences SEQ ID No. 25, SEQ ID No. 26, or SEQ ID No. 27.

In another of its aspects, the invention provides use of a HE capable of cleaving a non-native nucleotide sequence in a genome to manipulate a DNA sequence whose amino acid translation has at least 80% homology with the amino acid translation of the native target of the HE with the proviso that the DNA sequence is not the native nucleotide sequence.

For example, the HE may be PI-SceI and the native target SEQ. ID No. 3. In this case, the non-native target may be any one of the DNA sequences SEQ ID Nos. 4, 5, 6, 7, 8, 9, and 10. This use may be implemented in the treatment of osteoporosis.

The HE may be POLB HE and the native target SEQ. ID No. 13. The HE may be PRP8 HE and the native target SEQ. ID No. 17. In this case, the non-native target may be SEQ ID No. 14. This use may be implemented in the treatment of cancer, and in particular colon cancer or colorectal cancer.

The HE may be PRP8 HE and the native target SEQ. ID No. 17. In this case, the DNA sequence may be SEQ ID No. 18. This use may be implemented in the treatment of retinitis pigmentosa.

The HE may be Nostoc species PCC7120 HE and the native target SEQ ID No. 21. In this case, the non-native target may be any one of the DNA sequences SEQ ID No. 23 and SEQ ID No 24.

The manipulating of the DNA sequence may be selected from correcting the DNA sequence, disrupting the DNA sequence, inserting an exogenous DNA sequence, inducing homologous recombination, inducing non-homologous end joining. In the case of inserting an exogenous DNA sequence, the exogenous DNA sequence may be selected from a viral DNA sequence, a transposon, a gene, a regulatory element, and an intron.

The use of the invention may be implemented in crop improvement, animal model engineering, engineering of a cell line, engineering of induced pluripotent stem cells.

The invention also provides a method for the treatment of osteoporosis comprising administering to an individual in need of such treatment a pharmaceutical composition of the invention comprising PI-SceI.

The invention also provides a method for the treatment of cancer comprising administering to an individual in need of such treatment a pharmaceutical composition of the invention comprising POLB HE.

The invention also provides a method for the treatment of retinitis pigmentosa comprising administering to an individual in need of such treatment a pharmaceutical composition of the invention comprising PRP8 HE.

The invention also provides a method for the genetic manipulation of cyanobacteria comprising introducing into a cyanobacteria cell the HE Nostoc RNR or a nucleotide sequence encoding for the HE Nostoc RNR.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1a shows the alignment of the native target of the PI-SceI HE from S. cerevisiae with predicted targets in the human ATP6V1A1 gene and its homologs in the genomes of animal models. FIG. 1b shows results of an In vitro cleavage assay demonstrating that PI-SceI can cleave its predicted targets. UC=uncut plasmid, RE=plasmid cut with the XbaI restriction endonuclease only, HEN=plasmid cut with PI-SceI only, RE+HEN=plasmid cut with PI-SceI and XbaI.

FIG. 2a shows the design of an assay for the detection of cleavage of a nucleotide sequence by POLB HE of H. volvanii in which a PCR is preformed on H. volcanii individual colonies transformed with a plasmid encoding a POLB allele lacking the HE but carrying either the native target of the POLB HE or a homologous sequence from the human POLD1 gene. Cleavage of the sequence within the POLB allele by the HE leads to homing, the copying of the HE into the plasmid borne allele. The PCR can amplify either a short product in the absence of homing or a long product if homing has taken place. FIG. 2b shows nucleotide and amino-acid alignments of the target sequence from the H. volcanii POLB gene and the homologous human sequence from the POLD1 gene. FIG. 2c shows representative results from the colony PCR assay. A long PCR product indicates that homing occurred. FIG. 2d shows a graph showing the relative homing efficiency of the POLB HEN to the plasmid borne POLB allele carrying either native (wt) or human targets.

FIG. 3a shows a yeast assay for HE activity. A HE target site is inserted between truncated Ura3 repeats. Upon HE cleavage the truncated repeats recombine to reconstitute the metabolic marker, allowing the yeast to grow on the appropriate selective medium. FIG. 3b shows nucleotide alignment of the B. cinerea PRP8 HE-target and the homologous sequence from the human PRPF8 gene. FIG. 3c shows the relative activity of the B. cinerea PRP8 HE on its native target and on its human target in the PRPF8 gene (logarithmic scale).

FIG. 4a shows nucleotide alignment of the Nostoc species PCC7120 HE-target and the homologous sequence from Nostoc punctiforme and synechococcus. FIG. 4b shows the relative activity of the Nostoc species PCC7120 HE on its native target and on its targets in Nostoc punctiforme and synechococcus (logarithmic scale).

### EXPERIMENTAL RESULTS

### PI-SceI HE

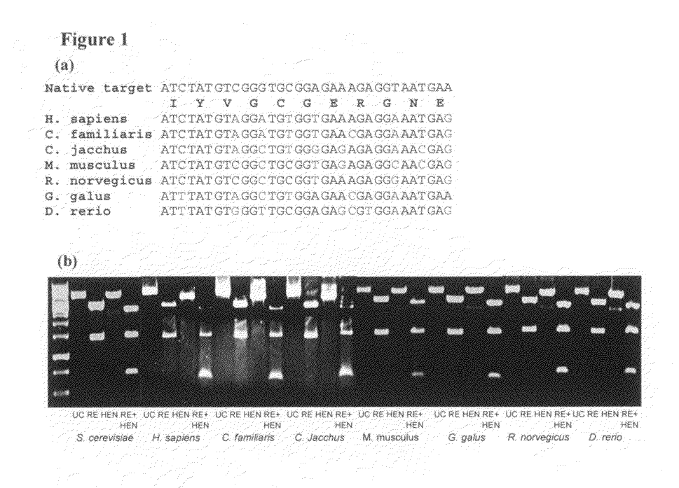

FIG. 1a shows the alignment of the native target of the PI-SceI HE in S. cerevisiae (SEQ ID 3) with a homolog of the native target in the human ATP6V1A1 gene (SEQ ID 4), as well as with homologs of the native target in the genome of six animals (SEQ ID 5 to 10). FIG. 1b (b) shows the results of an In vitro cleavage assay in which the ability of PI-SceI (obtained from New England Biolabs) to cleave each of the sequences shown in FIG. 1a was determined. The cleavage products were separated on an agarose gel and visualized by ethidium bromide staining. The leftmost lane in the gel of FIG. 1b is a I KD ladder of DNA which was obtained from Sigma. The results show that each of the sequences shown in FIG. 1a was cleaved by the PI-SceI HE.

### POLB HE

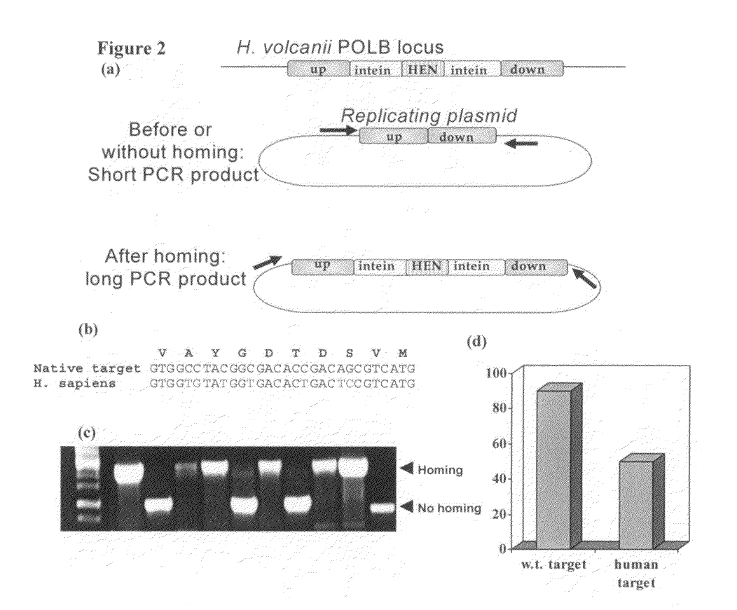

FIG. 2a shows the design of an assay to determine the ability of POLB HE to cleave various DNA sequences. A PCR was preformed on individual colonies of H. volcanii transformed with a plasmid carrying a POLB allele not encoding the POLB HE but encoding either the native target of the POLB HE (SEQ ID No. 13) or a homologous sequence from the human POLD1 gene. The PCR will amplify a short product in the absence of cleavage and a long product if cleavage has occurred. FIG. 2b shows the nucleotide and amino-acid alignments of the native target sequence from the H. volcanii POLB gene (SEQ ID No. 13) and the homologous human sequence from the POLD1 gene (SEQ ID No. 14). FIG. 2c shows representative results from the colony PCR assay. POLB HE was obtained by PCR of the genome of H. volcanii. A long PCR product indicates that cleavage took place. FIG. 2d shows a graph showing the relative homing efficiency of the POLB HE to the plasmid borne POLB allele carrying either the native (wt) or human target.

### PRP8 HE

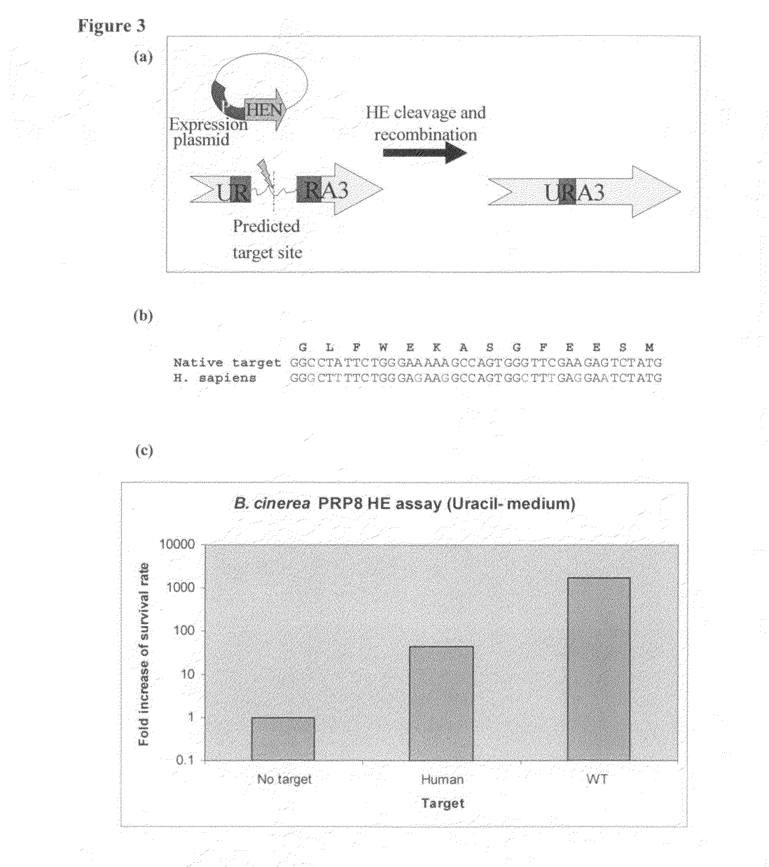

FIG. 3a shows the design of the yeast assay that was used to detect the ability of B. cinerea PRP8 HE to cleave various DNA sequences. A DNA sequence was inserted between truncated repeats of the metabolic marker Ura3 in S cervisiae. Upon cleavage by PRP8 HE, the truncated repeats recombine to reconstitute the metabolic marker, allowing the yeast to grow on a medium lacking Uracil. FIG. 3b shows the nucleotide alignment of the B. cinerea PRP8 HE-target (SEQ ID 17), a homologous sequence from the human PRPF8 gene (SEQ ID 18) and a homologous mouse PRPF8 gene sequence (SEQ ID No.19). FIG. 3c shows the relative activity of the B. cinerea PRP8 gene on its native target and on the human sequence from the PRPF8 gene, (logarithmic scale). The results show that PRP8 HE is capable of cleaving the non-native targets.

### NOSTOC

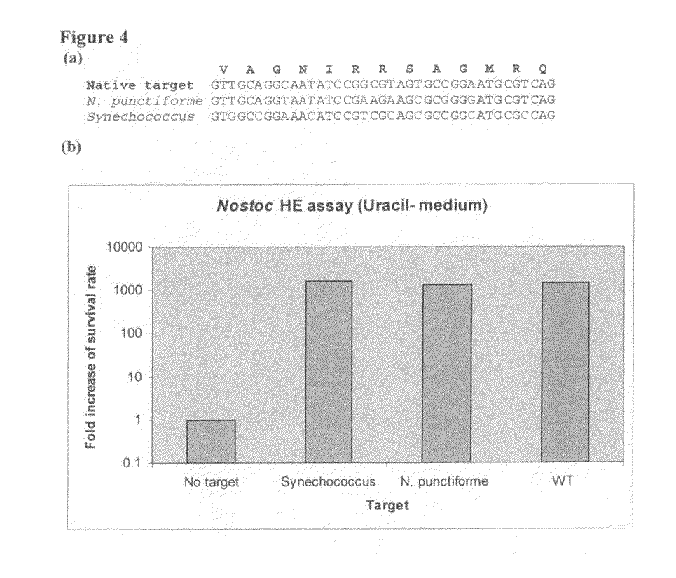

FIG. 4a shows nucleotide alignment of the Nostoc species PCC7120 HE-target (SEQ ID No. 22) and the homologous sequence from Nostoc punctiforme (SEQ ID No. 23) and synechococcus (SEQ ID No 24). FIG. 4b shows the relative activity of the Nostoc species PCC7120 HE on its native target and on its targets in Nostoc punctiforme and synechococcus (logarithmic scale).

1. A pharmaceutical composition comprising as an active ingredient either a homing endonuclease (HE) capable of cleaving a non-native target nucleotide sequence in a genome or a nucleotide sequence encoding for a HE capable of cleaving a target site in a non-native genome, together with a physiologically acceptable carrier.

2. The pharmaceutical composition according to claim 1 wherein the active ingredient is PI-SceI having the amino acid sequence SEQ ID No 2.

3. The pharmaceutical composition according to claim 1 wherein the active ingredient is POLB HE having the amino acid sequence SEQ ID NO. 12.

4. The pharmaceutical composition according to claim 1 wherein the active ingredient is PRP8 HE having the amino acid sequence SEQ ID No. 16 amino acid.

5. The pharmaceutical composition according to claim 1 wherein the active ingredient is a DNA sequence.

6. The pharmaceutical composition according to claim 1 wherein the active ingredient is an RNA sequence.

7. The pharmaceutical composition according to claim 5 wherein the DNA sequence is SEQ ID No. 1

8. The pharmaceutical composition according to claim 5 wherein the DNA sequence is SEQ ID No. 11

9. The pharmaceutical composition according to claim 5 wherein the DNA sequence is SEQ ID No. 15

10. The pharmaceutical composition according to claim 6 wherein the RNA sequence is SEQ ID No. 25

11. The pharmaceutical composition according to claim 6 wherein the RNA sequence is SEQ ID No. 26

12. The pharmaceutical composition according to claim 6 wherein the RNA sequence is SEQ ID No. 27

13. Use of a HE capable of cleaving a non-native nucleotide sequence in a genome to manipulate a DNA sequence whose amino acid translation has at least 80% homology with the amino acid translation of the native target of the HE with the proviso that the DNA sequence is not the native nucleotide sequence.

14. The use according to claim 13 wherein the HE is PI-SceI and the native target is SEQ. ID No. 3.

15. The use according to claim 13 wherein the HE is POLB HE and the native target is SEQ. ID No. 13.

16. The use according to claim 13 wherein the HE is PRP8 HE and the native target is SEQ. ID No. 17.

17. The use according to claim 14 wherein the DNA sequence is selected from SEQ ID Nos. 4, 5, 6, 7, 8, 9, and 10.

18. The use according to claim 15 wherein the DNA sequence is SEQ ID No. 14.

19. The use according to claim 16 wherein the DNA sequence is SEQ ID No. 18.

20. The use according to claim 13 wherein the HE is Nostoc species PCC7120 HE and the native target is SEQ ID No. 21.

21. The use according to claim 20 wherein the DNA sequence is selected from SEQ ID No. 23 and SEQ ID No 24.

22. The use according to claim 13 wherein the manipulating of the DNA sequence is selected from correcting the DNA sequence, disrupting the DNA sequence, inserting an exogenous DNA sequence, inducing homologous recombination, inducing non-homologous end joining.

23. The use according to claim 22 comprising inserting an exogenous DNA sequence wherein the exogenous DNA sequence is selected from a viral DNA sequence, a transposon, a gene, a regulatory element, and an intron.

24. The use according to claim 13 in crop improvement, animal model engineering, engineering of a cell line, engineering of induced pluripotent stem cells.

25. The use according to claim 14 in the treatment of osteoporosis.

26. The use according to claim 15 in the treatment of cancer.

27. The use according to claim 25 wherein the cancer is colon cancer or colorectal cancer.

28. The use according to claim 16 in the treatment of retinitis pigmentosa.

29. A method for the treatment of osteoporosis comprising administering to an individual in need of such treatment a pharmaceutical composition according to claim 2.

30. A method for the treatment of cancer comprising administering to an individual in need of such treatment a pharmaceutical composition according to claim 3.

31. A method for the treatment of retinitis pigmentosa comprising administering to an individual in need of such treatment a pharmaceutical composition according to claim 14.

32. A method for the genetic manipulation of cyanobacteria comprising introducing into a cyanobacteria cell the HE Nostoc RNR or a nucleotide sequence encoding for the HE Nostoc RNR.


SEQUENCE LISTING (27 sequences)

SEQ ID NO: 1 - DNA, 1362 bp, Homo sapiens
tgctttgccaagggtaccaatgttttaatggcggatgggtctattgaatgtattgaaaacattgaggttggtaataaggtcatgggtaaagatggcagacctcgtgaggtaattaaattgcccagaggaagagaaactatgtacagcgtcgtgcagaaaagtcagcacagagcccacaaaagtgactcaagtcgtgaagtgccagaattactcaagtttacgtgtaatgcgacccatgagttggttgttagaacacctcgtagtgtccgccgtttgtctcgtaccattaagggtgtcgaatattttgaagttattacttttgagatgggccaaaagaaagcccccgacggtagaattgttgagcttgtcaaggaagtttcaaagagctacccaatatctgaggggcctgagagagccaacgaattagtagaatcctatagaaaggcttcaaataaagcttattttgagtggactattgaggccagagatctttctctgttgggttcccatgttcgtaaagctacctaccagacttacgctccaattctttatgagaatgaccactttttcgactacatgcaaaaaagtaagtttcatctcaccattgaaggtccaaaagtacttgcttatttacttggtttatggattggtgatggattgtctgacagggcaactttttcggttgattccagagatacttctttgatggaacgtgttactgaatatgctgaaaagttgaatttgtgcgccgagtataaggacagaaaagaaccacaagttgccaaaactgttaatttgtactctaaagttgtcagaggtaatggtattcgcaataatcttaatactgagaatccattatgggacgctattgttggcttaggattcttgaaggacggtgtcaaaaatattccttctttcttgtctacggacaatatcggtactcgtgaaacatttcttgctggtctaattgattctgatggctatgttactgatgagcatggtattaaagcaacaataaagacaattcatacttctgtcagagatggtttggtttcccttgctcgttctttaggcttagtagtctcggttaacgcagaacctgctaaggttgacatgaatggcaccaaacataaaattagttatgctatttatatgtctggtggagatgttttgcttaacgttctttcgaagtgtgccggctctaaaaaattcaggcctgctcccgccgctgcttttgcacgtgagtgccgcggattttatttcgagttacaagaattgaaggaagacgattattatgggattactttatctgatgattctgatcatcagtttttgcttgccaaccaggttgtcgtccataat

SEQ ID NO: 2 - protein, 454 aa, Homo sapiens
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHN

SEQ ID NO: 3 - DNA, 33 bp, Homo sapiens
atctatgtcgggtgcggagaaagaggtaatgaa

SEQ ID NO: 4 - DNA, 33 bp, Homo sapiens
atctatgtaggatgtggtgaaagaggaaatgag

SEQ ID NO: 5 - DNA, 33 bp, Homo sapiens
atctatgtaggatgtggtgaacgaggaaatgag

SEQ ID NO: 6 - DNA, 33 bp, Homo sapiens
atctatgtaggctgtggggagagaggaaacgag

SEQ ID NO: 7 - DNA, 33 bp, Homo sapiens
atctatgtcggctgcggtgagagaggcaacgag

SEQ ID NO: 8 - DNA, 33 bp, Homo sapiens
atctatgtcggctgcggtgaaagagggaatgag

SEQ ID NO: 9 - DNA, 33 bp, Homo sapiens
atttatgtgggttgcggagagcgtggaaatgag

SEQ ID NO: 10 - DNA, 33 bp, Homo sapiens
atttatgtaggctgtggagaacgaggaaatgaa

SEQ ID NO: 11 - DNA, 1311 bp, Homo sapiens
agcgtgacgggagatcgcccggtcgtcgtcagagaccccggtgggactgttcgaatccttcctatcgaggacttgtttgcccgcggaacgactgaatctgaggtactcatcgctgccgacggggacgtcgtcgcaagtgccactcccgggaagactcgccgagcgctcgacgggtgggacgccctctctgtgaacgaagatggagaggcggagtggcaaccgattgcgcaggcgattcgccataacacagacaaaccggtggtgaacctccaacacaagttcggtgagtcgacgacgacgagagaccactcgtacgtcgtccccggtgaagacggcctcacaactgtctctccggacgacgtggcggagccgtatcgcgtctccggggtacccgatgtcgagcctgtcgagcaggtcgacgtctacgaggtccttcgtgggtacgaacgcgagtacgaggacggacggagcgtcgggagcgataattcgataacgaagcggaaacaaatccatgcggacgacgagtatgtctggttcggccacgagcaccaccgagacgtcgactcgaccgtcaaagtcaaacgattcgtcgatatcgacagcgaagatggtgcagcactcattcggctcctcggtgcgtacgtccctgaaggaagcgcctccactggcgagacggcgacgtcgaaattcggggccagtctcgctgaatccgaccgtgagtggctagcccaactccagcgagattactctcgactgttcgagaacacgaccgccggtatcattacgagcgaccgacgagcggagcgaaccgtcgagtatcaaacggacacaggcggtgcgtcggtcacgtacaatgacgagacgctgaaactgcagatgatgaacgaactcgctgctgtgttcttccgcgagttcgcagggcagacgtcgcgtggtaaacggatcccctcattcgtcttccaccttcccgaggagaagcaagacttgttcctgacgttgctcgtcgaaggcgatggatctcgcgaattcccacgatacaccgaagcgtacgcacagcgaaacttcgacttcgagacgacgagccgagaacttgctgccggtctctcgatgttgctcacgcaacgggggcaaaaacactcgctcaagtatcgggacagtaaagactcgtacactattcggacgtgtagcacctaccgggaaggccgagaccccgtgctgaccgaagccgaccacgacggctacgtgtacgacctgagcgtcgaagaaaacgaaaacttcgtcgacggtgttggaggtatcgtccttcacaac

SEQ ID NO: 12 - protein, 437 aa, Homo sapiens
SVTGDRPVVVRDPGGTVRILPIEDLFARGTTESEVLIAADGDVVASATPGKTRRALDGWDALSVNEDGEAEWQPIAQAIRHNTDKPVVNLQHKFGESTTTRDHSYVVPGEDGLTTVSPDDVAEPYRVSGVPDVEPVEQVDVYEVLRGYEREYEDGRSVGSDNSITKRKQIHADDEYVWFGHEHHRDVDSTVKVKRFVDIDSEDGAALIRLLGAYVPEGSASTGETATSKFGASLAESDREWLAQLQRDYSRLFENTTAGIITSDRRAERTVEYQTDTGGASVTYNDETLKLQMMNELAAVFFREFAGQTSRGKRIPSFVFHLPEEKQDLFLTLLVEGDGSREFPRYTEAYAQRNFDFETTSRELAAGLSMLLTQRGQKHSLKYRDSKDSYTIRTCSTYREGRDPVLTEADHDGYVYDLSVEENENFVDGVGGIVLHN

SEQ ID NO: 13 - DNA, 30 bp, Homo sapiens
gtggcctacggcgacaccgacagcgtcatg

SEQ ID NO: 14 - DNA, 30 bp, Homo sapiens
gtggtgtatggtgacactgactccgtcatg

SEQ ID NO: 15 - DNA, 2433 bp, Homo sapiens
tgtcttgcgtgggatacgaaactcttacgatacgacggtaccgacgtcgtcgttcaggacgttaaggaaggggatctacttctcggtccggacggtggaccccgtcgggcattcaatatcgttagtggtaaagacaggctttaccgaatcaaggttggttcacgtaaagaagacctcgttgtcacgggaaaccatattctagtcttgcaccgggagaaagggcacggcaacgtctacgacggaccatccgttgggggaaatcgtcaacgtttcgtcgatcaactcggtgatctgccagtaccgagctctaaccctgctgatgctacacgtcctaataatctcacgaaggttcgtccagacttcttagcagctctaaagagtgctattgcttgggcattgaacgccgagcgtgggaagaagggtgctgacactattcgcaacacactcaacggcacaaccgggatcacttcacgccaggagagctatatcgtcaacattcccgttggaaaaggcacgagagccgagtatgccacattcgcctggggaaacccagatcggacagtgaaaggccacgctaaacacccccctgaattcttcccgaccaaggaggatgcattttctgcttctgttgctaagagcaggcagattcatgacaaaggtgacgtgactctggctacactacgccgtcgtttccttgacaaatcctcagatgggaaagggggggaacttcgaattgacacaggcttgcctaatatgttcctcctttggaatgcaaacggcgcgaacctcaagattcgcgtgtattgctctcgcaactataccaagtacgggcgatcttatacatttccatctctaccagacataaacctgtctgaggctggttctgatgactcagatgacaacgaagagaccgagaacgaagacgacgagaccgaggacgaagacgacgagagagaagaaacattgactctccagaatttccagagtaccgcctcccgagacgtttcctccgccgaacgctacgacacggttctaatgacggcaacccaattcgcagcactggacgaaaacgagagatctaaatacaggctctttcgttctcctggattcgagttgcctgagcaagatgtcccagtcaacccctactttcttggtctatggttaggagatggcagccgtagctcaaccacaatcttcagtaatcacgagcaggaagtaagggagtttctcatttctcacgctgctgagctagaccttcacctcgtttggcacgggaacctctcgtatgcgactgttggcaggactcgcattgctaatagacccttaccgaaagccaacatcgacgtcgtcgatcgtccatcaagacgcttttctcgacagaccatcaaaaagcaacgcgaggctgcagagctaccctcaaggcccgctccagctgtggccaatttgaagcatggtctcaatagtagtgttccgaattccccacaacgtcgcttacgacaacgtatcgacgacgtcgatgtgcagaaccttgtcgatggtatggataacttaacttcatcacctattccttcgtcaccacccgttatcccagctgaatcgattccaactgaggctctgccacagcttagatccgatagaagcattatggatatggctggtccgtctgtagttcctgaagaacctgtagatgtcaataacctgcccgaggatgaggaagatgagtttgatatggatcttattgagacaatgagcgacgacgaggacgatgtcaccgagtaccaagttgaaaatgacgaaggatccaacgtcggagctggggatagtaatctctcggacgactctgtctctcaacgacgaattcatcgcctgcaaagtggacgtcgagcatatggtgacttgcagcctgaagagcaagaccagctcctgagtcagataatcgacacggtcgattctccagtcggttcttcgttcgatccttcgaccgacaagaaacacatcccatcgatttatatgaagaacacgcgcgaagttcgtctcgctgttcttgctggtctgattgattctgacggctggtacgtgtatcccgagaatatgcttgggttcgctcagagcgagatctggcataagactctcttctgggacgttgtcgcgctggcaagatcattgggtctcagcgtctggaccacgagacgtatgatgtgggttccgagccattcacgaaaaactcctatgctcgtagcccagatgtttggcaacgtgaaagaagtgccctgtttgctcctgcgcaagaagggatctgagcgttatattccgcaaatgcacagctttatgatcaaagacatcacccttgaatcagaggcaacgaattgggctgggttccgagtcgataaagaccagctgtatcttcgtcacgattaccttgtacttcataac

SEQ ID NO: 16 - protein, 811 aa, Homo sapiens
CLAWDTKLLRYDGTDVVVQDVKEGDLLLGPDGGPRRAFNIVSGKDRLYRIKVGSRKEDLVVTGNHILVLHREKGHGNVYDGPSVGGNRQRFVDQLGDLPVPSSNPADATRPNNLTKVRPDFLAALKSAIAWALNAERGKKGADTIRNTLNGTTGITSRQESYIVNIPVGKGTRAEYATFAWGNPDRTVKGHAKHPPEFFPTKEDAFSASVAKSRQIHDKGDVTLATLRRRFLDKSSDGKGGELRIDTGLPNMFLLWNANGANLKIRVYCSRNYTKYGRSYTFPSLPDINLSEAGSDDSDDNEETENEDDETEDEDDEREETLTLQNFQSTASRDVSSAERYDTVLMTATQFAALDENERSKYRLFRSPGFELPEQDVPVNPYFLGLWLGDGSRSSTTIFSNHEQEVREFLISHAAELDLHLVWHGNLSYATVGRTRIANRPLPKANIDVVDRPSRRFSRQTIKKQREAAELPSRPAPAVANLKHGLNSSVPNSPQRRLRQRIDDVDVQNLVDGMDNLTSSPIPSSPPVIPAESIPTEALPQLRSDRSIMDMAGPSVVPEEPVDVNNLPEDEEDEFDMDLIETMSDDEDDVTEYQVENDEGSNVGAGDSNLSDDSVSQRRIHRLQSGRRAYGDLQPEEQDQLLSQIIDTVDSPVGSSFDPSTDKKHIPSIYMKNTREVRLAVLAGLIDSDGWYVYPENMLGFAQSEIWHKTLFWDVVALARSLGLSVWTTRRMMWVPSHSRKTPMLVAQMFGNVKEVPCLLLRKKGSERYIPQMHSFMIKDITLESEATNWAGFRVDKDQLYLRHDYLVLHN

SEQ ID NO: 17 - DNA, 42 bp, Homo sapiens
ggcctattctgggaaaaagccagtgggttcgaagagtctatg

SEQ ID NO: 18 - DNA, 42 bp, Homo sapiens
gggcttttctgggagaaggccagtggctttgaggaatctatg

SEQ ID NO: 19 - DNA, 42 bp, Homo sapiens
ggtctgttctgggagaaagccagtggctttgaggagtccatg

SEQ ID NO: 20 - DNA, 1221 bp, Homo sapiens
tgtctcccagaagatgctttagttcatactgctaaaggtttagttccgattcgtgacgtgcaagttggtgacttggtacagactcctttaggattccggcgagttgttgataaattcgaccaagggtttcaagatgtctacgaaattgaaactaatgccacctaccccagggcgactttaaaccacagacaagctgtgttagaagatgctaagggtggcattgtctggaaacatattgctagtttagaagcaggcgatcgcctactgcacaacaagcaagttctacctggtacagttacccatctacccgccgattttacagaatctcgtccctcccacagccgcacagctaaatccttcgttgttcctgaactaacggcggaagtggcttggctcatcgggtttacccacggtgatggctatgtagctcttgggcgcaataaatacgataaaccatacggtcgtgttgagtggtcaatgaacagcctagatgctgaagtcacaagtagaatacaagctaaaatcgatgctgctttggctttatttggcttgagtgctgttcatagcattaccaaaggtgagaacactgccaaatcaatctgttcatcaattcggctagctgagtatttccatcgtcacatcaaacagcccaatattcccctgacagttcctagttttatcttgcaaggttcagtagatatcagggctgcatatctagctggcttaatggatagtgatggtgctgttaataaccgtcctcctcacctaattacctcagtttatcggtcatttattcgacaagtaagtgtagttctatctagcctgggtattgctgggagacttaccacaacttacccgcaaaattcaaattggcaagtcaaatacaacttgacaattccagcgctaaaggagcgttacaatgccctgatttcgccgcattcagctaaaggtgaactacgtcaaggcctgaaaatgtatggttttaccgttcctggtgcagtcatgcgggaaacttacacctacagcgaaatgcgcgagatgggatttcaaggctcccgtactgtagatgctaattacgaacgctacgttgctgaggcagatatttccttagatattcctgtcacagtgaaaggattaggcagttacgatcatgttcaaacctatgatatagaagtcgatgaagctcattgcttctactgcgacggctatctcacacataac

SEQ ID NO: 21 - protein, 407 aa, Homo sapiens
CLPEDALVHTAKGLVPIRDVQVGDLVQTPLGFRRVVDKFDQGFQDVYEIETNATYPRATLNHRQAVLEDAKGGIVWKHIASLEAGDRLLHNKQVLPGTVTHLPADFTESRPSHSRTAKSFVVPELTAEVAWLIGFTHGDGYVALGRNKYDKPYGRVEWSMNSLDAEVTSRIQAKIDAALALFGLSAVHSITKGENTAKSICSSIRLAEYFHRHIKQPNIPLTVPSFILQGSVDIRAAYLAGLMDSDGAVNNRPPHLITSVYRSFIRQVSVVLSSLGIAGRLTTTYPQNSNWQVKYNLTIPALKERYNALISPHSAKGELRQGLKMYGFTVPGAVMRETYTYSEMREMGFQGSRTVDANYERYVAEADISLDIPVTVKGLGSYDHVQTYDIEVDEAHCFYCDGYLTHN

SEQ ID NO: 22 - DNA, 39 bp, Homo sapiens
gttgcaggcaatatccggcgtagtgccggaatgcgtcag

SEQ ID NO: 23 - DNA, 39 bp, Homo sapiens
gttgcaggtaatatccgaagaagcgcggggatgcgtcag

SEQ ID NO: 24 - DNA, 39 bp, Homo sapiens
gtggccggaaacatccgtcgcagcgccggcatgcgccag

SEQ ID NO: 25 - DNA, 1362 bp, Homo sapiens
ugcuuugccaaggguaccaauguuuuaauggcggaugggucuauugaauguauugaaaacauugagguugguaauaaggucauggguaaagauggcagaccucgugagguaauuaaauugcccagaggaagagaaacuauguacagcgucgugcagaaaagucagcacagagcccacaaaagugacucaagucgugaagugccagaauuacucaaguuuacguguaaugcgacccaugaguugguuguuagaacaccucguaguguccgccguuugucucguaccauuaagggugucgaauauuuugaaguuauuacuuuugagaugggccaaaagaaagcccccgacgguagaauuguugagcuugucaaggaaguuucaaagagcuacccaauaucugaggggccugagagagccaacgaauuaguagaauccuauagaaaggcuucaaauaaagcuuauuuugaguggacuauugaggccagagaucuuucucuguuggguucccauguucguaaagcuaccuaccagacuuacgcuccaauucuuuaugagaaugaccacuuuuucgacuacaugcaaaaaaguaaguuucaucucaccauugaagguccaaaaguacuugcuuauuuacuugguuuauggauuggugauggauugucugacagggcaacuuuuucgguugauuccagagauacuucuuugauggaacguguuacugaauaugcugaaaaguugaauuugugcgccgaguauaaggacagaaaagaaccacaaguugccaaaacuguuaauuuguacucuaaaguugucagagguaaugguauucgcaauaaucuuaauacugagaauccauuaugggacgcuauuguuggcuuaggauucuugaaggacggugucaaaaauauuccuucuuucuugucuacggacaauaucgguacucgugaaacauuucuugcuggucuaauugauucugauggcuauguuacugaugagcaugguauuaaagcaacaauaaagacaauucauacuucugucagagaugguuugguuucccuugcucguucuuuaggcuuaguagucucgguuaacgcagaaccugcuaagguugacaugaauggcaccaaacauaaaauuaguuaugcuauuuauaugucugguggagauguuuugcuuaacguucuuucgaagugugccggcucuaaaaaauucaggccugcucccgccgcugcuuuugcacgugagugccgcggauuuuauuucgaguuacaagaauugaaggaagacgauuauuaugggauuacuuuaucugaugauucugaucaucaguuuuugcuugccaaccagguugucguccauaau

SEQ ID NO: 26 - DNA, 1311 bp, Homo sapiens
agcgugacgggagaucgcccggucgucgucagagaccccggugggacuguucgaauccuuccuaucgaggacuuguuugcccgcggaacgacugaaucugagguacucaucgcugccgacggggacgucgucgcaagugccacucccgggaagacucgccgagcgcucgacgggugggacgcccucucugugaacgaagauggagaggcggaguggcaaccgauugcgcaggcgauucgccauaacacagacaaaccgguggugaaccuccaacacaaguucggugagucgacgacgacgagagaccacucguacgucguccccggugaagacggccucacaacugucucuccggacgacguggcggagccguaucgcgucuccgggguacccgaugucgagccugucgagcaggucgacgucuacgagguccuucguggguacgaacgcgaguacgaggacggacggagcgucgggagcgauaauucgauaacgaagcggaaacaaauccaugcggacgacgaguaugucugguucggccacgagcaccaccgagacgucgacucgaccgucaaagucaaacgauucgucgauaucgacagcgaagauggugcagcacucauucggcuccucggugcguacgucccugaaggaagcgccuccacuggcgagacggcgacgucgaaauucggggccagucucgcugaauccgaccgugaguggcuagcccaacuccagcgagauuacucucgacuguucgagaacacgaccgccgguaucauuacgagcgaccgacgagcggagcgaaccgucgaguaucaaacggacacaggcggugcgucggucacguacaaugacgagacgcugaaacugcagaugaugaacgaacucgcugcuguguucuuccgcgaguucgcagggcagacgucgcgugguaaacggauccccucauucgucuuccaccuucccgaggagaagcaagacuuguuccugacguugcucgucgaaggcgauggaucucgcgaauucccacgauacaccgaagcguacgcacagcgaaacuucgacuucgagacgacgagccgagaacuugcugccggucucucgauguugcucacgcaacgggggcaaaaacacucgcucaaguaucgggacaguaaagacucguacacuauucggacguguagcaccuaccgggaaggccgagaccccgugcugaccgaagccgaccacgacggcuacguguacgaccugagcgucgaagaaaacgaaaacuucgucgacgguguuggagguaucguccuucacaac

SEQ ID NO: 27 - DNA, 2433 bp, Homo sapiens
ugucuugcgugggauacgaaacucuuacgauacgacgguaccgacgucgucguucaggacguuaaggaaggggaucuacuucucgguccggacgguggaccccgucgggcauucaauaucguuagugguaaagacaggcuuuaccgaaucaagguugguucacguaaagaagaccucguugucacgggaaaccauauucuagucuugcaccgggagaaagggcacggcaacgucuacgacggaccauccguugggggaaaucgucaacguuucgucgaucaacucggugaucugccaguaccgagcucuaacccugcugaugcuacacguccuaauaaucucacgaagguucguccagacuucuuagcagcucuaaagagugcuauugcuugggcauugaacgccgagcgugggaagaagggugcugacacuauucgcaacacacucaacggcacaaccgggaucacuucacgccaggagagcuauaucgucaacauucccguuggaaaaggcacgagagccgaguaugccacauucgccuggggaaacccagaucggacagugaaaggccacgcuaaacaccccccugaauucuucccgaccaaggaggaugcauuuucugcuucuguugcuaagagcaggcagauucaugacaaaggugacgugacucuggcuacacuacgccgucguuuccuugacaaauccucagaugggaaagggggggaacuucgaauugacacaggcuugccuaauauguuccuccuuuggaaugcaaacggcgcgaaccucaagauucgcguguauugcucucgcaacuauaccaaguacgggcgaucuuauacauuuccaucucuaccagacauaaaccugucugaggcugguucugaugacucagaugacaacgaagagaccgagaacgaagacgacgagaccgaggacgaagacgacgagagagaagaaacauugacucuccagaauuuccagaguaccgccucccgagacguuuccuccgccgaacgcuacgacacgguucuaaugacggcaacccaauucgcagcacuggacgaaaacgagagaucuaaauacaggcucuuucguucuccuggauucgaguugccugagcaagaugucccagucaaccccuacuuucuuggucuaugguuaggagauggcagccguagcucaaccacaaucuucaguaaucacgagcaggaaguaagggaguuucucauuucucacgcugcugagcuagaccuucaccucguuuggcacgggaaccucucguaugcgacuguuggcaggacucgcauugcuaauagacccuuaccgaaagccaacaucgacgucgucgaucguccaucaagacgcuuuucucgacagaccaucaaaaagcaacgcgaggcugcagagcuacccucaaggcccgcuccagcuguggccaauuugaagcauggucucaauaguaguguuccgaauuccccacaacgucgcuuacgacaacguaucgacgacgucgaugugcagaaccuugucgaugguauggauaacuuaacuucaucaccuauuccuucgucaccacccguuaucccagcugaaucgauuccaacugaggcucugccacagcuuagauccgauagaagcauuauggauauggcugguccgucuguaguuccugaagaaccuguagaugucaauaaccugcccgaggaugaggaagaugaguuugauauggaucuuauugagacaaugagcgacgacgaggacgaugucaccgaguaccaaguugaaaaugacgaaggauccaacgucggagcuggggauaguaaucucucggacgacucugucucucaacgacgaauucaucgccugcaaaguggacgucgagcauauggugacuugcagccugaagagcaagaccagcuccugagucagauaaucgacacggucgauucuccagucgguucuucguucgauccuucgaccgacaagaaacacaucccaucgauuuauaugaagaacacgcgcgaaguucgucucgcuguucuugcuggucugauugauucugacggcugguacguguaucccgagaauaugcuuggguucgcucagagcgagaucuggcauaagacucucuucugggacguugucgcgcuggcaagaucauugggucucagcgucuggaccacgagacguaugauguggguuccgagccauucacgaaaaacuccuaugcucguagcccagauguuuggcaacgugaaagaagugcccuguuugcuccugcgcaagaagggaucugagcguuauauuccgcaaaugcacagcuuuaugaucaaagacaucacccuugaaucagaggcaacgaauugggcuggguuccgagucgauaaagaccagcuguaucuucgucacgauuaccuuguacuucauaac